(19) United States
(12) Patent Application Publication
(10) Pub. No.: US 2022/0051753 A1
(43) Pub. Date: Feb. 17, 2022

(54) METHOD AND DEVICE FOR ASCERTAINING AN RNA SEQUENCE

(71) Applicant: Robert Bosch GmbH, Stuttgart (DE)

(72) Inventors: Rolf Backofen, Freiburg (DE); Frank Hutter, Freiburg Im Breisgau (DE); Frederic Runge, Freiburg (DE)

(21) Appl. No.: 17/354,992

(22) Filed: Jun. 22, 2021

(30) Foreign Application Priority Data
Aug. 14, 2020 (DE) 102020210357.7

(51) Int. Cl. G16B 15/10

(52) U.S. Cl. CPC G16B 15/10

(57) ABSTRACT

A method for creating a strategy, which is configured to determine a placement of nucleotides within a primary RNA structure as a function of a detail of a predefined secondary structure. The method includes the following steps: initializing the strategy; providing a task representation, the task representation including structural restrictions of the secondary RNA structure and sequential restrictions of the primary RNA structure; determining a primary candidate RNA sequence with the aid of the strategy as a function of the task representation; adapting the strategy with the aid of a reinforcement learning algorithm in such a way that a total loss is optimized.

### CROSS REFERENCE

The present application claims the benefit under 35 U.S.C. § 119 of German Patent Application No. DE 102020210357.7 filed on Aug. 14, 2020, which is expressly incorporated herein by reference in its entirety.

### FIELD

The present invention relates to a method for ascertaining an RNA sequence with the aid of a trained strategy, a training device, a computer program, and a machine-readable memory medium.

### BACKGROUND INFORMATION

The design of RNA molecules has awoken interest in medicine, synthetic biology, biotechnology, and bioinformatics in recent times, since it has been shown that many functional RNA molecules participate in regulatory processes for transcription, epigenetics, and translation. Since the function of RNA is dependent on its structural properties, the RNA design problem is to find an RNA sequence which satisfies the given structural restrictions.

The paper Runge et al., “Learning to design RNA,” in International Conference on Learning Representations, 2019. Retrievable online:

https://openreview.net/forum?id=ByfyHh05tQ describes an algorithm for the RNA design problem using “deep reinforcement learning” to train a policy network to design an entire RNA sequence sequentially which corresponds to a specified target structure.

### SUMMARY

However, present formulations of the RNA design significantly restrict their solution space in that they require a structural priority on the entire molecule or on at least the full form of the desired molecule.

The present invention may have the advantage over the related art that a larger search space may be explored, thus a much more versatile candidate sequence having practical relevance may be created/found, which was previously not findable by computer science. Up to this point, it has not been possible to deal with unbalanced parentheses and partial structures. Using the present invention, it is possible to define within a “design task” and find solutions.

Furthermore, the method in accordance with the present invention is capable of transferring the learned knowledge to tasks of earlier RNA design formulations. RNA sequences may thus be found more efficiently. The partial RNA design according to the present invention may be understood as the super problem of inverse RNA design and inverse RNA design with sequence specifications.

In a first aspect, the present invention relates to a computer-implemented method for creating a strategy it, which is configured to determine a placement of nucleotides within a primary RNA structure as a function of a detail of a specified secondary structure.

In accordance with an example embodiment of the present invention, the method includes the following steps: initializing the strategy. The strategy may be implemented, for example, by a neural network. For this purpose, the strategy may be initialized in that, for example, weights of the neural network are set randomly.

This is followed by providing a task representation τ, task representation τ including structural restrictions w of the secondary RNA structure and sequential restrictions ψ of a primary RNA structure. This is followed by determining a primary candidate RNA sequence ϕ with the aid of strategy π as a function of task representation τ, the positions of the primary RNA structure of candidate RNA sequence ϕ successively being filled with the ascertained nucleotides of strategy π with the aid of strategy π. This is followed by ascertaining a sequence loss L_{ψ} of candidate RNA sequence ϕ to sequential restrictions ψ;

applying an (RNA) folding algorithm F to candidate RNA sequence ϕ.

This is followed by ascertaining a structure loss L_{w} between folded structure F(ϕ) and predefined structural restrictions w; ascertaining a total loss L_{τ} as a function of sequence loss L_{ψ} and structure loss L_{w}.

This is followed by adapting strategy π with the aid of a reinforcement learning algorithm in such a way that total loss L_{τ} is optimized.

It is provided that the detail is a function of a parameter, this parameter also being optimized upon the optimization of the strategy.

In a second aspect, the present invention relates to a method for determining an RNA sequence ϕ given a partial secondary structure and a partial primary structure of the RNA with the aid of learned strategy π, which is configured to determine a placement of nucleotides of the RNA as a function of a detail of the secondary structure. In accordance with an example embodiment of the present invention, the method includes the following steps: providing a task representation τ and successively determining a candidate RNA sequence ϕ with the aid of the strategy as a function of the details of task representation τ.

In further aspects, the present invention relates to a device and a computer program which are each configured to carry out the above methods and a machine-readable memory medium on which this computer program is stored.

Specific example embodiments of the present invention are explained in greater detail hereinafter with reference to the figures.

### BRIEF DESCRIPTION OF THE DRAWINGS

FIG. 1 shows a schematic illustration of an RNA design problem.

FIG. 2 schematically shows an exemplary embodiment of a specific embodiment of the present invention.

FIG. 3 shows a schematic illustration of a hyperparameter optimization of a reinforcement learning algorithm.

FIG. 4 shows a table including possible hyperparameters.

FIG. 5 shows a possible structure of a training device.

### DETAILED DESCRIPTION OF EXAMPLE EMBODIMENTS

In its most fundamental structural form, RNA is a sequence of the four nucleotides adenine (A), guanine (G), cytosine (C), and uracil (U). This nucleotide sequence is referred to as the RNA sequence or primary structure.

While the RNA sequence is used as a blueprint, the functional structure of the RNA molecule is determined by the folding, which translates the RNA sequence into its 3D tertiary structure. The intrinsic thermodynamic properties of the sequence determine the resulting folding. The hydrogen bonds which are formed between two corresponding nucleotides represent one of the driving forces in the thermodynamic model and strongly influence the tertiary structure. The structure which includes these hydrogen bonds is generally referred to as the secondary structure of the RNA.

The problem of finding an RNA sequence which folds into a desired secondary structure is known as the RNA design problem or RNA inverse folding.

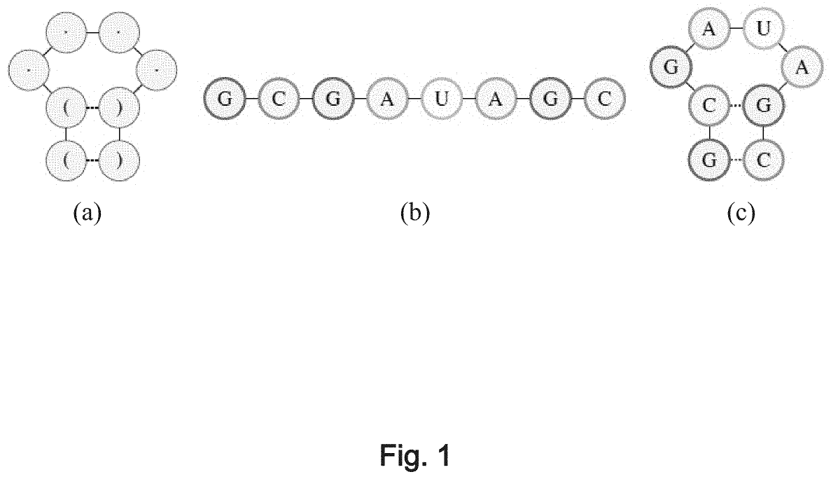

FIG. 1 schematically shows an illustration of the RNA design problem using a folding algorithm F and a point-parenthesis notation. In consideration of the desired RNA secondary structure, which is represented in point-parenthesis notation (a), the object is to design an RNA sequence (b) which folds into desired secondary structure (c).

In the following, a “partial RNA design” is to be defined and a specific embodiment of the present invention is to be explained to integrate both sequence and structural features into a simple, shared task representation, among other things, to assist a knowledge transfer over various RNA design tasks.

RNA design considers two search spaces: The sequence space includes chains of nucleotides NϵΦ:={A,C;G;U}, while the structure space is made up of sequences of typical secondary structural features BϵΩ:={.,(,)}. It is to be noted that the typical point-parenthesis notation according to Ivo Hofacker, Walter Fontana, Peter Stadler, Sebastian Bonhoeffer, Manfred Tacker, and Peter Schuster, “Fast Folding and Comparison of RNA Secondary Structures,” Chemical Monthly, 125:167-188, 02 1994, is used here.

An RNA folding algorithm F translates between these spaces by mapping an RNA sequence ϕϵΦ¹={A, C, G, U}^{l} of length l to its corresponding secondary structure [symbol] (ϕ)ϵΩ^{l}={., (,)}^{l}.

RNA design addresses the inverse process: given a sequence wϵΩ^{l} of secondary structural features, it is the goal to find an RNA sequence ϕ so that it fulfills the equation w= [symbol] (ϕ).

Additional sequence restrictions ψϵΨ^{l}:=(Φ∪{N})^{l} may be used to exclude parts of the solution space, which makes the RNA design into an NP-hard problem, cf. https://www.liebertpub.com/doi/full/10.1089/cmb.2019.0420.

Partial RNA design expands this formulation by permitting unrestricted domains in the structure space, which may result in RNA design tasks which contain unbalanced parentheses, and opens the door for exploration by computer-assisted methods. Partial RNA design may be formally defined as follows:

[symbol] is an RNA folding algorithm and wϵ(Ω∪{B})^{l}={., (,),B}^{l} is a sequence of structure restrictions of length 1, which restricts the space of valid RNA secondary structures to Ω_{w}^{l}⊆Ω^{l} and ψϵΨ^{l} denotes a sequence of nucleotide restrictions, which restricts the space of valid RNA sequences to Φ_{w}^{l}⊆Φ^{l}, the goal of partial RNA design is to find an RNA sequence which satisfies the following equation: ϕϵΦ_{ψ}^{l}∧ [symbol] (ϕ)ϵΩ_{w}^{l}.

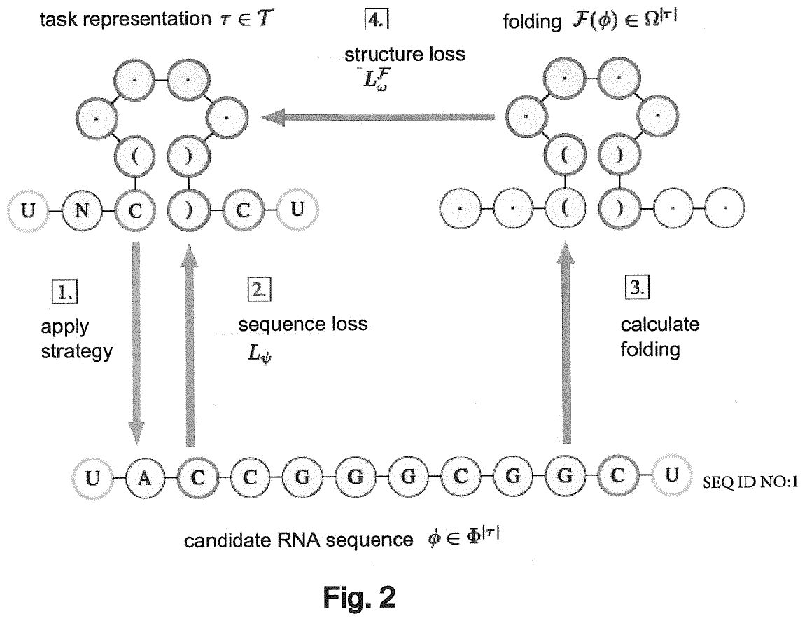

Since it is the goal to predict reasonable RNA sequences for any arbitrary setting of structure and sequence features, including partially and completely defined structure restrictions, a simple but general description of RNA design tasks is to be used hereinafter to enable a knowledge transfer between various RNA design tasks. Therefore, the two sequences of structure and sequence restrictions w,ψ are combined to form a shared representation τϵT*:={A,C,G,U,N,.,(,)}*. The shared representation is also referred to hereinafter as task representation τ, cf. FIG. 2.

In addition, a function C: (Ω∪{B})×(Φ∪{N})→(Φ∪Ω∪{N}) is defined which maps each point (also referred to as a position hereinafter) of RNA sequence iϵ{1, . . . , l} from restrictions w⁽ⁱ⁾, ψ⁽ⁱ⁾ in a single representation on either w⁽ⁱ⁾ for w⁽ⁱ⁾≠B and ψ⁽ⁱ⁾=N or on ψ⁽ⁱ⁾ in all other cases.

In addition, a preprocessing step may be carried out, which fills paired positions, of which only one interacting nucleotide is known, using its complementary pairing partner (according to a Watson-Crick base pair scheme). Positions at which the pairing partner is not to be established trivially, may be skipped and it is continued at the next paired position.

In reinforcement learning (RL), an agent acts via perception and action with a dynamic environment. In each step of the interaction, the agent receives an indication of the present state of the environment and selects an action on the basis of this observation. The action changes the state of the environment, and the value of this transition is communicated to the agent as a scalar reward signal. The end goal of the agent is to maximize a long-term amount of the reward signal. Since the actions may influence the state transitions and thus all following rewards, achieving an optimum behavior may be a very difficult task. In particular, it is not stated to the agent which action would have been in its best interest in the long term, and it thus searches by systematic testing, guided by a variety of different algorithms, for example, temporal difference learning (TD), Q learning, or policy gradient methods.

An RL algorithm for the inverse RNA folding was provided by Runge et al. (cf. above section related art), which is used as the foundation of the present invention. In RL, a policy if of the agent is approximated using an artificial neural network which outputs, for example, a distribution via actions. In contrast, the environment may be completely defined by a decision process which provides an array of available actions, an array of states, a reward function, and a state transition probability matrix. Reference is made to the approach described by Runge et al. for modeling the partial RNA designed as a reinforcement learning problem: The formulation of the states based on the available molecular features and actions correspond to the placement of nucleotides. As soon as all positions have been assigned nucleotides, the environment calculates the reward on the basis of the Hamming distance, which is communicated to the agent to update its model. The strategy is then set with the aid of RL algorithms in such a way that this minimizes the Hamming distance. The precise formulation of the decision process and architecture of the policy network may be optimized jointly together with further parameters.

Most inverse RNA folding algorithms use a structural loss function L_{w}(F(ϕ)) to quantify the distance between target structure w and structure F(ϕ), which results from the folding of an RNA sequence ϕ. An optimal candidate structure, also called minimizer ϕ*, has the smallest value of the loss function and corresponds to a solution of the inverse RNA folding problem for predefined target structure w.

A typical loss function is Hamming distance d_{H}. For the partial RNA structure, the desired structure may be only partially known and the solution space may additionally be restricted in the sequence space. Therefore, the loss formulation provided above by Runge et al. is adapted to consider only the positions of the designed candidate solution which are restricted either in the structure space, in the sequence space, or both. Whenever a location is unrestricted, it is excluded from the calculation of the distance and thus excluded from the calculation of the loss function. This may be formalized with the aid of an indicator function 1(C\hat{ }(i)), which returns the value 1 for a sequence of restrictions C of length l if position i is restricted, and 0 if position i is not restricted.

The loss for partially defined restrictions may be expressed by summing the Hamming distances between the restricted position of the sequence of nucleotide restrictions ψ and the corresponding positions of designed candidate solution ϕ; and between the restricted positions of the sequence of the structure restrictions w and the corresponding positions of folding F(ϕ). In a sequence of nucleotide boundary conditions Ψ of length l, this results in a sequence loss L_{ψ}(ϕ):

$${L}_{ψ} ( ϕ ) = {\sum }_{i = 1}^{l} 1 ( {ψ}^{( i )} ) * {d}_{H} ( {ψ}^{( i )} , {ϕ}^{( i )} )$$

Accordingly, structure loss L_{W}^{F}(ϕ) may be formulated as follows:

$${L}_{w}^{F} ( ϕ ) = {\sum }_{i = 1}^{l} 1 ( {w}^{( i )} ) * {d}_{H} ( {w}^{( i )} , {F ( ϕ )}^{( i )} )$$

Total loss L_{τ} for a specific RNA task representation τ and a specific designed candidate solution ϕ may then be defined as:

L_{T}=(L_{W}^{F}(ϕ)+L_{ψ}(ϕ))/|τ|

Minimizer ϕ* is then given by: ϕ*=argmin_{Φ} L_{τ}

The incorporation of sequence restrictions may be achieved in various ways. Therefore, a dimension for three different approaches for the generative design of candidate solutions in the shared configuration space was provided, which are described in the following paragraphs.

Naïve approach: For the naïve approach, the agent predicts a nucleotide for each position of an RNA design task τ, including the sequence parts.

Replacing approach: The replacing approach follows the same strategy as the naïve approach, but as soon as all positions are filled with nucleotides, the sequence parts of task representation τ replace the corresponding predicted parts of the candidate solution before the designed RNA sequence is rewarded.

Partial approach: In the third approach, the sequence domains of task representation τ are completely ignored, and the agent only predicts nucleotides for the structure parts and the unrestricted positions.

RL learning is used to update parameters λ of the neural network of strategy π^{λ} (policy network). The precise architecture of these networks is optimized jointly with the formulation of the decision process, the training hyperparameters, the training data distribution, the training teaching plan, and the algorithm used for the sequence design. The policy gradient method, proximal policy optimization (PPO), is used for updating parameters λ of a given policy network π. Runge et al. has previously shown that the meta-learning of an RNA design policy outperforms other learning strategies with respect to speed and accuracy, the present invention now adapting this strategy to solving the partial RNA design problem. In particular, each sampled RL algorithm initially learns an RNA design policy across thousands of local RNA design tasks (alternating sequence and structure motives). For a new, previously unseen design task, candidate solutions are then sampled from the strategy without further parameter updates.

Carrying out RL learning methods may react very sensitively to decisions with respect to the parameters of the agent, the environment, and the training parameters, and the formulation of an RL algorithm for a new problem is a difficult and protracted process; since there is no experience about which design decisions could provide the best results. An automation of the RL formulation could drastically cut down on this process. To solve this problem, an automated approach of reinforced learning (autoRL) is provided, which automatically selects the best learning environment for the reinforcement to solve the partial RNA design problem, in view of an extensive configuration space. In particular, a meta-learning process is defined for the shared optimization of the formulation of the RL algorithm: in the outer loop, the iterative meta-learning samples of a configuration which define an RL algorithm, which is then used to learn RNA design rules in the inner loop. The rule resulting therefrom is evaluated at a validation data set and the meta-learner observes the validation loss to update its own model accordingly. The goal of the meta-learner is to minimize the validation loss in that it learns to test out better configurations with each observation, while the learner attempts to maximize its reward for each task of the validation set. An approach of the present invention may be formally formulated as follows.

A is a set of algorithms for the generative RNA sequence design, E is a set of RL learning environments, N is a set of RL learning agents, Dₜᵣₐᵢₙ is an array of training data, and C is a set of training curriculums, which define a configuration space: Θ:=A×E×N×Dₜᵣₐᵢₙ×C.

The cost function for a specific configuration θϵΘ of entire validation set Dᵥₐₗ may then be described as:

L_{D}_{ᵥₐₗ}(A^{(θ)},E^{(θ)},N^{(θ)},C^{(θ)},Dₜᵣₐᵢₙ^{(θ)}).

The goal for the partial RNA design is to train a meta-learner L on the training data (training data+validation set), so that it finds an optimal configuration θ*ϵΘ which minimizes the cost function:

θ*=argmin_{Θ}L_{D}_{ᵥₐₗ}(A^{(θ)},E^{(θ)},N^{(θ)},C^{(θ)},Dₜᵣₐᵢₙ^{(θ)})

The search space thus represents an expanded configuration space of Runge et al. and contains five new dimensions.

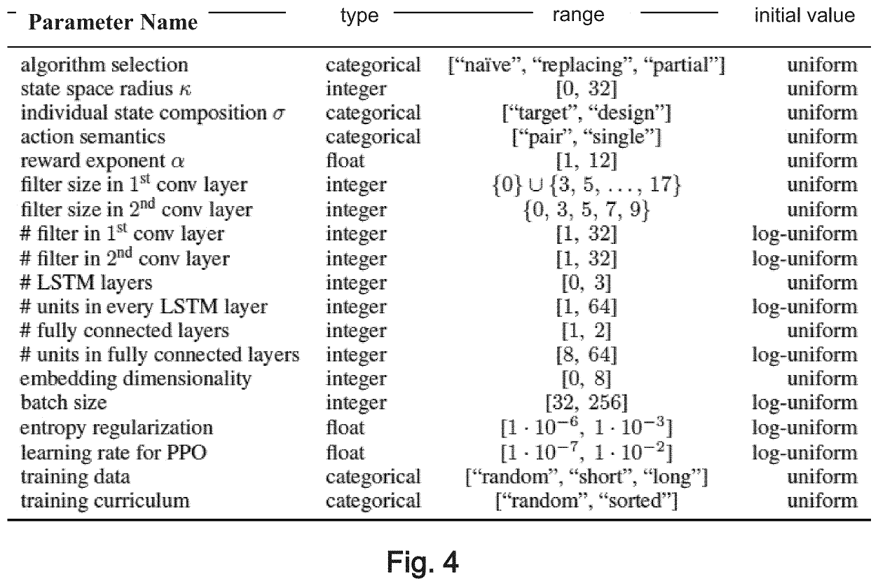

The configuration space includes four components: Decisions about the agents, the environment, the training data, and the algorithm described hereinafter for the sequence design. The table in FIG. 4 gives an overview.

Agent subspace: Each agent of agent subspace A is defined by a specific architecture of the policy network and the selected values for an array of training hyperparameters, which regulate the optimization and regularization. Except for minor changes, the agent subspace is primarily adapted to the parameters described under Runge et al. The architecture subspace is constructed as follows: (1) the task representation is either coded, a differentiation being made between paired positions, unpaired positions, and positions including specific nucleotides or wildcard symbols, or being processed by an optional embedding layer which converts the symbol-based representation into a numerical representation learnable for each side. Furthermore, (2) an optional CNN including at most two layers followed by (3) an optional LSTM including at most three layers may be selected on the embedding layer. Finally, (4) a flat network including one or two layers is added, which outputs the distribution via actions. This parameterization covers a broad range of possible neural architectures and keeps the dimension of the search space relatively small. The search space for the neural architecture for the policy network is shown in FIG. 4. Each path in the diagram of FIG. 4 corresponds to a specific architecture. The performance of neural networks is strongly dependent on the selection of the hyperparameters. Preferably, some of the parameters of PPO which are used for the formation of the network are incorporated into the shared configuration space: the learning rate, the batch size, and the strength of the entropy regularization.

Environment subspace: Environment subspace E is defined by selection of values for the parameterized decision process D_{θ}:=(S_{θ}; A_{θ}; R_{θ}; P_{θ}). The specific values for the parameters of the decision process are optimized jointly with the other parameters of configuration space θ. In particular, the state formulation is optimized by the number of the positions which are centered symmetrically around the present position, using state radius K, and the exact configuration of each state via the individual state configuration. In addition, the influence of pair predictions and the parameters for the design of the reward may be analyzed using the action semantics parameter. Finally, the transition dynamic range is a function of multiple parameter decisions of different subspaces and is defined accordingly.

Training data subspace: The training data determine which task distribution is inspected during the training and which states are explored. Preferably, three training sets of different task distributions are incorporated in the shared configuration space, which are accessible via the training data parameters. Since different curriculums may result in different performances of a specific ML algorithm, furthermore a training curriculum parameter may be introduced to select between a random curriculum or a sorted curriculum of the training data with respect to the task length.

Algorithm selection subspace: It is possible to select here between the above-explained approaches: the naïve approach, the replacement approach, and the partial approach.

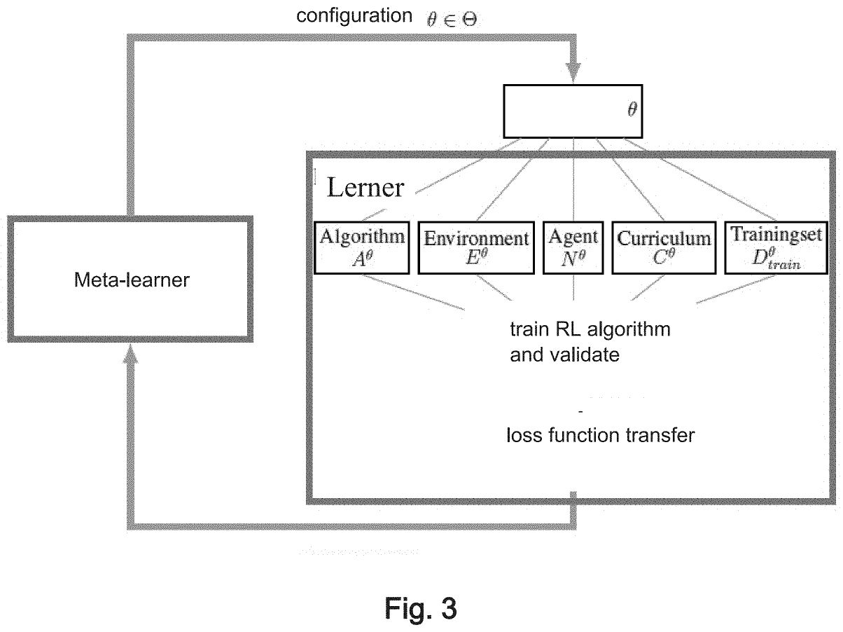

In this section, the procedure is described in greater detail to automatically select the best RL algorithm for the partial RNA design from the shared configuration space. Optimizer BOHB is preferably used for the meta-learning, in particular as the meta-learner according to FIG. 3. BOHB was selected because it deals with mixed discrete/continuous search spaces, uses parallel resources, and moreover may advantageously evaluate approximations of the objective function to accelerate the optimization. These so-called low fidelity approximations may be achieved in various ways, for example, by limiting the training time, the number of independent repetitions of the assessments, or by using only fractions of the available data. The training time is preferably limited for the sampled RL algorithm.

Data sets: The ultimate goal of the present approach is to design RNA candidates for any type of constraint setting in the sequence and the structure space in that knowledge is transferred between various RNA design tasks. To correctly optimize the listed design decisions with respect to this goal, training and validation data sets are necessary, including tasks which contain unbalanced parentheses.

Target function: Although RL is known to supply noisy or unreliable results in individual optimization passes, preferably only a single meta-optimization pass and a single validation set are used. To consider noisy results of the optimization process, preferably three loss formulations are previously examined for the optimization method: (1) the number of the unsolved goals, (2) the sum of the mean distances, and (3) the sum of the minimum distances. Based on preliminary results, it has been shown that variant (3), the sum of the minimum distances, is advantageous as the goal for the optimization. However, variant (1) may also result in good results. The number of the unsolved goals is particularly preferably minimized during the meta-optimization process according to variant (1).

Budgets: It has been found to be advantageous that candidate solutions for 100 previously unseen local RNA design tasks of the validation set from the learned RNA designed guideline having fixed parameters are used. To approach the performance with different reliability, the wall clock time for the training procedure may be limited. Each RL algorithm would be assessed for 60 seconds on the tasks of the validation set. Finally, the established configuration was selected for the assessment of the various test sets.

Parameter importance: To analyze the importance of individual parameters, we use the functional ANOVA (fANOVA) framework, which is based on random forests. The five most important parameters in the meta-optimization were the algorithm selection parameter, the action semantics parameter, the number of the LSTM levels, the learning rate, and the state radius (in order of importance).

Overall, the design decisions result in a 19-dimensional design space, which includes a broad spectrum of neural architectures to formulate the agent (including elements of repeating neural networks (RNNs) and convolutional neural networks (CNNs)), a plurality of different environmental formulations, three different training data distributions, two training curriculums, three different algorithms for the generative design of RNA sequences and training hyperparameters. The complete list of the parameters, their types, ranges, and the priorities are listed in FIG. 4.

An efficient Bayesian optimization method may be used for optimizing the RL formulation, cf., for example, Stefan Falkner, Aaron Klein, and Frank Hutter, “BOHB: Robust and efficient hyperparameter optimization at scale,” in Jennifer Dy and Andreas Krause, editors, Proceedings of the 35th International Conference on Machine Learning, volume 80 of Proceedings of Machine Learning Research, pages 1437-1446, Stockholmsmässan, Stockholm Sweden, 10-15 Jul. 2018. PMLR. Retrievable online: http://proceedings.mlr.press/v80/falkner18a.html.

In RL, the strategy of the agent is approximated by an artificial deep neural network which outputs a distribution over the possible actions, a representation of the present state. In contrast, the environment may be completely defined by the formulation of the decision process D: =(S; A; R; P), which includes an array of states S, an array of available actions A, a reward function R, which was already introduced above, and a state transition probability matrix P. The following paragraphs describe the various components which model the partial RNA design as a decision process.

The state space is represented as follows. In each time step t=0; 1; 2,::::, T, T representing the terminal time step of the episodic interaction between the agent and the environment, the environment supplies a state st which instructs the agent when learning a strategy. To provide local items of information to the agent, a (2κ+1) gram may be used, which is centered around the t-th point of task representation τ, κ being a hyperparameter which is referred to as the state radius. To be able to construct this centered n-gram at all locations, pad characters (“#”) may be added at the beginning and at the end of the task task representation).

The action space is made up of the available four nucleotides. It is conceivable that Watson-Crick base pairs are also used (AU, UA, GC, CG) for paired positions in task representation τ.

The state transition dynamics may be modeled as follows. In each time step t, the state is set to a fixed (2κ+1) gram, and following states are defined by deterministic transitions over individual positions of the task representation. The transition dynamics may vary as a function of the selection of the action semantics and the selected algorithm for generating candidate solution ϕ and on the selection of the state configuration, and would be implemented accordingly.

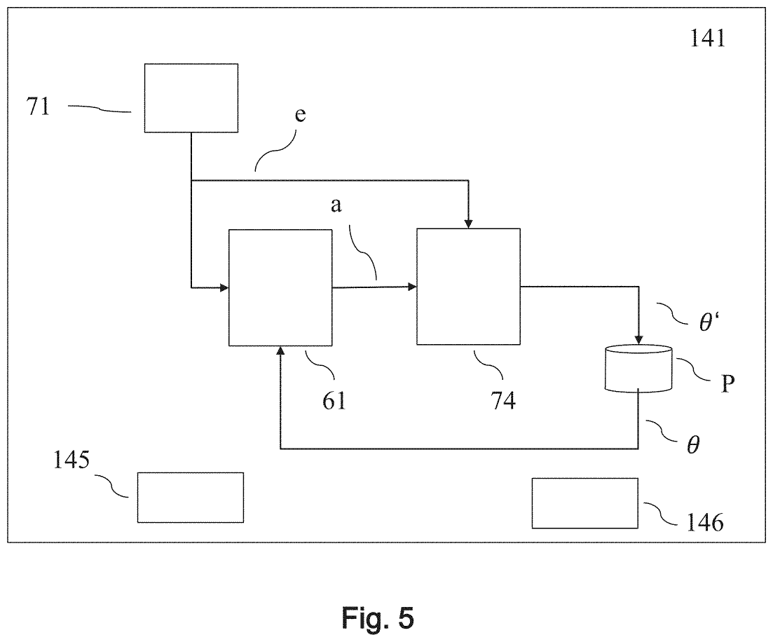

FIG. 5 schematically shows a training device 141 including a provider 71, which provides training sequences e from a training data set. These are supplied to monitoring unit 61 to be trained, which ascertains total losses a therefrom. Total losses a and training sequences e are supplied to an evaluator 74, which ascertains parameters θ′ of the strategy which are transmitted to parameter memory P and replace parameters θ therein.

The method carried out by training device 141 may be stored, implemented as a computer program, on a machine-readable memory medium 146 and executed by a processor 145.

What is claimed is:

1. A computer-implemented method for creating a strategy, which is configured to determine a placement of nucleotides within a primary RNA structure as a function of a detail of a predefined secondary structure, the method comprising the following steps:
initializing the strategy;
providing a task representation, the task representation including structural restrictions of a secondary RNA structure and sequential restrictions of the primary RNA structure;
determining a primary candidate RNA sequence using the strategy as a function of the task representation, points of the primary RNA structure of the candidate RNA sequence successively being filled with ascertained nucleotides of the strategy with the aid of strategy;
ascertaining a sequence loss of the candidate RNA sequence to the sequential restrictions;
applying a folding algorithm to the candidate RNA sequence to provide a folded structure;
ascertaining a structure loss between the folded structure and the predefined structural restrictions;
ascertaining a total loss as a function of the sequence loss and the structure loss;
adapting the strategy using a reinforcement learning algorithm in such a way that the total loss is optimized.

2. The method as recited in claim 1, wherein the detail is a function of a parameter, the parameter also being optimized during the optimization of the strategy.

3. The method as recited in claim 1, wherein an indicator function is used to ascertain the sequence loss and/or the structure loss.

4. The method as recited in claim 1, wherein the sequence loss is ascertained using a Hamming distance.

5. The method as recited in claim 1, wherein the total loss is divided by a number of the restrictions of the task representation.

6. The method as recited in claim 1, wherein a meta-learner is used to optimize hyperparameters of the reinforcement learning algorithm.

7. The method as recited in claim 1, wherein the meta-learner is a BOHB.

8. A method for determining an RNA sequence given a partial secondary structure and a partial primary structure of the RNA using a learned strategy, the learned strategy configured to determine a placement of nucleotides within a primary RNA structure as a function of a detail of a predefined secondary structure, the learned strategy being determined by initializing the strategy, providing a task representation, the task representation including structural restrictions of a secondary RNA structure and sequential restrictions of the primary RNA structure, determining a primary candidate RNA sequence using the strategy as a function of the task representation, points of the primary RNA structure of the candidate RNA sequence successively being filled with ascertained nucleotides of the strategy with the aid of strategy, ascertaining a sequence loss of the candidate RNA sequence to the sequential restrictions, applying a folding algorithm to the candidate RNA sequence to provide a folded structure, ascertaining a structure loss between the folded structure and the predefined structural restrictions, ascertaining a total loss as a function of the sequence loss and the structure loss, and adapting the strategy using a reinforcement learning algorithm in such a way that the total loss is optimized, the method comprising the following steps:
providing the task representation; and
successively determining a candidate RNA sequence using as a function of details of the task representation.

9. A device configured to create a strategy, which is configured to determine a placement of nucleotides within a primary RNA structure as a function of a detail of a predefined secondary structure, the device configured to:
initialize the strategy;
provide a task representation, the task representation including structural restrictions of a secondary RNA structure and sequential restrictions of the primary RNA structure;
determine a primary candidate RNA sequence using the strategy as a function of the task representation, points of the primary RNA structure of the candidate RNA sequence successively being filled with ascertained nucleotides of the strategy with the aid of strategy;
ascertain a sequence loss of the candidate RNA sequence to the sequential restrictions;
apply a folding algorithm to the candidate RNA sequence to provide a folded structure;
ascertain a structure loss between the folded structure and the predefined structural restrictions;
ascertain a total loss as a function of the sequence loss and the structure loss; and
adapt the strategy using a reinforcement learning algorithm in such a way that the total loss is optimized.

10. A non-transitory machine-readable memory medium on which is stored a computer program for creating a strategy, which is configured to determine a placement of nucleotides within a primary RNA structure as a function of a detail of a predefined secondary structure, the computer program, when executed by a computer, causing the computer to perform the following steps:
initializing the strategy;
providing a task representation, the task representation including structural restrictions of a secondary RNA structure and sequential restrictions of the primary RNA structure;
determining a primary candidate RNA sequence using the strategy as a function of the task representation, points of the primary RNA structure of the candidate RNA sequence successively being filled with ascertained nucleotides of the strategy with the aid of strategy;
ascertaining a sequence loss of the candidate RNA sequence to the sequential restrictions;
applying a folding algorithm to the candidate RNA sequence to provide a folded structure;
ascertaining a structure loss between the folded structure and the predefined structural restrictions;
ascertaining a total loss as a function of the sequence loss and the structure loss;
adapting the strategy using a reinforcement learning algorithm in such a way that the total loss is optimized.


SEQUENCE LISTING (1 sequence)

SEQ ID NO: 1 - RNA, 12 nt, Artificial Sequence
  Synthetic Polynucleotide
uaccgggcggcu